(19) United States
(12) Patent Application Publication
(10) Pub. No.: US 2014/0194722 A1
(43) Pub. Date: Jul. 10, 2014

(54) LESION DIAGNOSIS APPARATUS AND METHOD

(71) Applicant: Samsung Electronics Co., Ltd., Suwon-si (KR)

(72) Inventors: Jae-Cheol LEE, Seoul (KR); Kyoung-Gu WOO, Seoul (KR); Yeong-Kyeong SEONG, Suwon-si (KR)

(73) Assignee: Samsung Electronics Co., Ltd., Suwon-si (KR)

(21) Appl. No.: 14/140,724

(22) Filed: Dec. 26, 2013

(30) Foreign Application Priority Data
Jan. 10, 2013 (KR) 10-2013-0002994

(51) Int. Cl. A61B 5/00

(52) U.S. Cl. CPC A61B 5/4887; A61B 5/0059
USPC 600/407

(57) ABSTRACT

A lesion diagnosis apparatus and method for diagnosing images in real time to notify a user that a lesion is detected and providing the user with information about the detected lesion, thereby enhancing accuracy and convenience in a diagnosis. The lesion diagnosis apparatus includes a diagnoser configured to diagnose lesions in images received from a probe; and a guide information provider configured to notify a user that a lesion is detected and to provide guide information, when the lesion is detected.

### CROSS-REFERENCE TO RELATED APPLICATION

This application claims the benefit under 35 U.S.C. §119(a) of Korean Patent Application No. 10-2013-0002994, filed on Jan. 10, 2013, the entire disclosure of which is incorporated herein by reference for all purposes.

### BACKGROUND

1. Field

The following description relates to a lesion diagnosis apparatus and method.

2. Description of the Related Art

Computer Aided Diagnosis (CAD) is a diagnosis technique that performs a lesion diagnosis through a lesion search, feature extraction, and feature classification by analyzing medical images received from an ultrasound probe. In CAD, it is valuable to receive a medical image containing a lesion quickly and to perform a lesion diagnosis quickly and precisely. An ultrasound imaging device scans a lesion from a medical image using a probe, captures a suspected area to contain a lesion as a still image, and stores the captured image in a Picture Archiving and Communication System (PACS). A CAD apparatus receives an image stored in the PACS and performs a lesion diagnosis on the received image through lesion search, feature extraction, and feature classification. If a health care professional fails to capture an area as a still image using a probe, the CAD apparatus cannot perform a lesion diagnosis on the area, and thus, a user may fail to recognize a lesion existing in the area.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a lesion diagnosis apparatus is provided, and the lesion diagnosis apparatus includes a diagnoser configured to diagnose lesions in images received from a probe; and a guide information provider configured to notify a user that a lesion is detected and to provide guide information, when the lesion is detected.

The diagnoser may be further configured to diagnose a non-overlapping area in a image using at least one of coordinates, a measuring range, a measured angle, or a measurement direction of the probe.

The lesion diagnosis apparatus may include a store configured to store lesion information when the lesion is detected, and the lesion information comprises at least one of features of the lesion, diagnosis of the lesion, location information of the lesion, or information about the movement of the probe after the lesion is detected.

The guide information may guide the probe toward a location of the lesion, and the guide information may include at least one of a moving path, a moving distance, a moving direction, or a measured angle of the probe.

The guide information may be provided as at least one of an audio signal, a visual signal, or a tactile signal

The guide information provider may notify the user that a lesion is detected by generating a detection signal and the detection signal may include at least one of a visual signal, an audio signal, or a tactile signal.

In response to a determination being made that the detected lesion is different from a previously detected lesion, the guide information provider may be configured to generate a detection signal.

The lesion diagnosis apparatus may include an input means configured to receive a confirmation from the user that the detected lesion is recognized; and the lesion information further comprises indication whether the user has recognized the detected lesion.

The guide information provider may be configured to generate a detection signal when a lesion that has not been recognized by the user is re-detected.

The guide information provider may be configured to generate a detection signal when a lesion is re-detected and its diagnosis is different from a previous diagnosis.

The features of the lesion may comprise at least one of shape of the lesion, boundary of the lesion, posterior acoustic shadow of the lesion, echo pattern, microlobulations, hyperechoic halo, hypoechoic nodule, duct extension, compressibility, branching pattern, or density of the lesions.

The diagnoser may be further configured to exclude the detected lesion that has been recognized by the user, from an area to be diagnosed.

In another general aspect, a lesion diagnosis method including diagnosing lesions in images received from a probe; generating, at a guide information provider, a detection signal to notify a user that the lesion is detected; and providing guide information to guide the probe toward a location of the detected lesion.

The diagnosing of lesions may include diagnosing a non-overlapping area in an image by using at least one of coordinates, a measuring range, a measured angle, or a measurement direction of the probe.

The lesion diagnosis method may include storing lesion information when the lesion is detected, and the lesion information may include at least one of features of the lesion, diagnosis of the lesion, location information of the lesion, or information about the movement of the probe after the lesion is detected.

The guide information may include at least one of a moving path, a moving distance, a moving direction, or a measured angle of the probe.

Providing of the guide information may comprise providing the guide information as at least one of an audio signal, a visual signal, or a tactile signal.

The detection signal may include at least one of a visual signal, an audio signal, or a tactile signal.

The generating of the detection signal may include generating a detection signal when the detected lesion is different from a previously detected lesion.

The lesion diagnosis method may include receiving a confirmation from the user that the detected lesion is recognized, and the lesion information may include indication whether the user has recognized the detected lesion.

The generating of the detection signal may include generating a detection signal when a lesion that has been detected but not recognized by the user is re-detected.

The generating of the detection signal may include generating a detection signal when a lesion is re-detected and its diagnosis is different from a previous diagnosis.

The features of the lesion may include at least one of shape of the lesion, boundary of the lesion, posterior acoustic shadow of the lesion, echo pattern, microlobulations, hyperechoic halo, hypoechoic nodule, duct extension, compressibility, branching pattern, or density of the lesions.

In another general aspect, a lesion diagnosis method including diagnosing lesions, in real-time, for images received from a probe; and generating, at a guide information provider, a detection signal to notify a user that the lesion is detected.

The diagnosing lesions in real-time may include diagnosing lesions in a first image received from the probe; identifying a non-overlapping area between the first image and a second image received from the probe; and diagnosing lesions only in the non-overlapping area.

The non-overlapping area may be identified using at least one of coordinates, a measuring range, a measured angle, or a measurement direction of the probe.

The lesion diagnosis method may include providing guide information to guide the probe toward a location of the detected lesion; and receiving a confirmation from the user that the detected lesion is recognized.

The providing of the guide information comprises continuing to provide guide information until the user confirms that the detected lesion is recognized.

The lesion diagnosis method may include storing lesion information when the lesion is detected; and the lesion information may comprise at least one of features of the lesion, diagnosis of the lesion, location information of the lesion, information about the movement of the probe after the lesion is detected, or indication whether the user has recognized the detected lesion.

In another general aspect, a lesion diagnosis apparatus including a diagnoser configured to receive a image captured by a probe at a first point in time, and to detect lesions in the image at a second point in time; and a guide information provider configured to provide guide information to compensate for the movement of the probe between the first point in time and the second point in time.

The guide information may guide the probe toward a location of the lesion, and the guide information may include at least one of a moving path, a moving distance, a moving direction, or a measured angle of the probe.

The guide information may be provided as at least one of an audio signal, a visual signal, or a tactile signal.

The guide information provider may be configured to generate an increasing volume of a sound or frequency as the user moves the probe closer to the detected lesion.

The guide information provider may be configured to increase the intensity of vibration of the probe as the probe moves closer to the detected lesion.

Other features and aspects will be apparent from the following detailed description, the drawings, and the claims.

### BRIEF DESCRIPTION OF THE DRAWINGS

FIGS. 1 and 2 are diagrams illustrating examples of configurations of a lesion diagnosis apparatus.

FIGS. 3A to 3C are diagrams illustrating examples for explaining examples of location information of a lesion, information about movement of a probe, and an overlapping area.

FIG. 4 is a diagram illustrating an example for generating a detection signal and providing guide information.

FIG. 5 is a diagram illustrating an example of a method for generating a detection signal.

FIG. 6 is a diagram illustrating an example of a method for providing guide information.

FIG. 7 is a diagram illustrating an example of a method for generating a detection signal when a lesion is re-detected.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will be apparent to one of ordinary skill in the art. Also, descriptions of well-known functions and constructions may be omitted for increased clarity and conciseness.

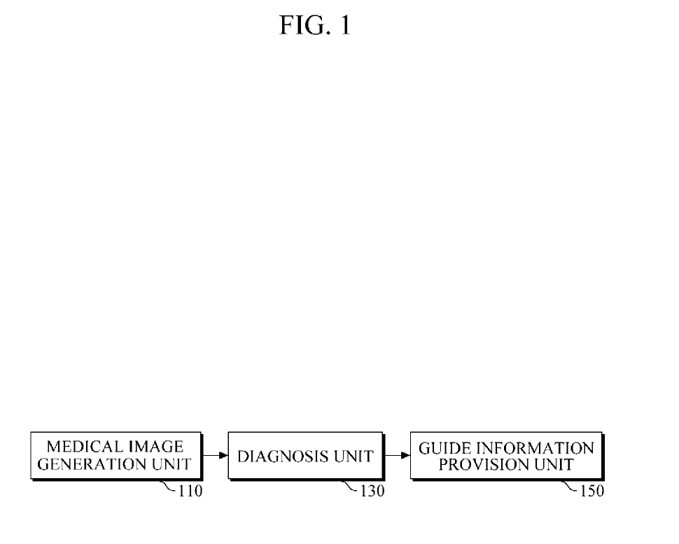

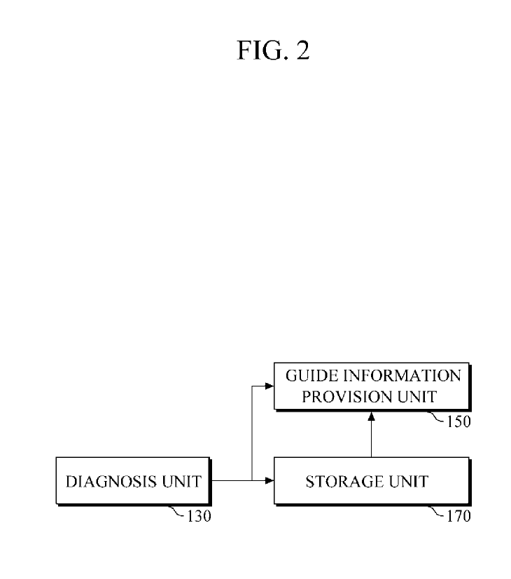

FIGS. 1 and 2 are diagrams illustrating examples of configurations of a lesion diagnosis apparatus. Referring to FIG. 1, a medical image generation unit 110 includes a probe and an image processing means. The probe transmits an ultrasound signal to a body part or tissue of a patient to obtain a medical image and receives an ultrasound echo signal reflected from the body part or tissue. The image processing means converts the ultrasound echo signal received from the probe to an electronic signal to generate a digital image. If a user moves the probe along the surface of the body part of the patient, the image processing means generates medical images in real time. As a non-exhaustive example only, the probe may include at least one of an accelerometer, a pressure sensor, or a tilt sensor (for example, a gyroscope sensor). The sensors may be installed in the inside or on the outside of the probe, and a medical image generated by the probe may be a two-dimensional (2D) image or a three-dimensional (3D) image.

A diagnosis unit 130 may diagnose lesions in medical images, which are received from the probe as the probe moves along the surface of the body part of the patient. Medical image generated by the medical image generation unit 110 is input to the diagnosis unit 130, and the diagnosis unit 130 outputs a result of a diagnosis by performing lesion detection, lesion feature extraction, and lesion classification. The diagnosis unit 130 makes a lesion diagnosis on each frame of a received medical image or on every predetermined number of frames that are sampled. As a non-exhaustive example only, one or more diagnosis unit 130 may be provided and the medical images generated by the medical image generation unit 110 may be distributed to the multiple diagnosis units 130 to be diagnosed concurrently.

If the diagnosis unit 130 detects a legion, a guide information providing unit 150 may generate a detection signal to inform a user that the lesion is detected. As a non-exhaustive example only, the detection signal may be generated as a signal that a user is able to recognize by hearing, such as a sound or voice with a specific pattern. As another non-exhaustive example only, the detection signal may be a signal that a user is able to recognize by touching, such as probe vibration. As another non-exhaustive example only, the detection signal may be displayed in a display device (not illustrated) as a message notifying the user that the lesion is detected or may be a visual signal that a user is able to recognize, such as a turning on and off of an additionally-installed light (not illustrated). As another non-exhaustive example only, the detection signal may be generated in the form of Picture in Picture (PIP). For example, the display device (not illustrated) may be displaying a medical image received from the medical image generation unit 110 in real time and an image with the detected lesion may be displayed on one side of the displayed medical image as a Picture in Picture (PIP). The examples of the detection signal generated by the guide information providing unit 150 are only non-exhaustive illustrations of the detection signal, and other forms of detection signal are considered to be well within the scope of the present disclosure.

Referring to FIG. 2, the lesion diagnosis apparatus may further include a storage unit 170. When the diagnosis unit 130 detects a lesion, the storage unit 170 may store lesion information including, but not limited to, at least one of features of the lesion, a diagnosis of the lesion, location information of the lesion, or information about movement of a probe after the lesion is detected. The features of the lesion may include various features necessary to identify the lesion from a medical image and make a diagnosis of the lesion, such as, for example, shape, boundary, posterior acoustic shadow, echo pattern, microlobulations, hyperechoic halo, hypoechoic nodule, duct extension, compressibility, branching pattern, and density of the lesions. The diagnosis may include whether the detected lesion is malignant or benign.

The location information of the lesion may include, but is not limited to, at least one of coordinates of a detected location of the lesion, a measured angle of the lesion, or a measured direction of the lesion. The information about movement of a probe may include, but is not limited to, at least one of a moving path, a moving distance, a moving angle, or a moving direction of the probe after the lesion is detected. The lesion location information and the information about movement of the probe may be measured using sensors, such as, for example, a tilt sensor and an accelerometer.

The lesion diagnosis apparatus may further include an input means (not illustrated) to receive a user's confirmation that the detected lesion is recognized. As a non-exhaustive example only, the input means may be a mouse, a keyboard, or an additional button installed in the probe. As another non-exhaustive example only, the input means (not illustrated) may be combined with a display device. The display device can encompass any combination of display region, gesture capture region, a touch sensitive display, and/or a configurable area. The display device can be embedded in the lesion diagnosis apparatus or may be an external peripheral device that may be attached and detached from the lesion diagnosis apparatus. The display device may be a single-screen or a multi-screen display. A single physical screen can include multiple displays that are managed as separate logical displays permitting different content to be displayed on separate displays although part of the same physical screen.

As another non-exhaustive example only, the probe may serve as the input means (not illustrated). A user may, for example, confirm that the detected lesion is recognized by moving the probe in a predetermined pattern. For example, the user may not move the probe for a predetermined length of time after detecting the lesion.

If a user confirms that the detected lesion is recognized, the storage unit 170 stores the user's confirmation. In one non-exhaustive example, based on the lesion information stored in the storage unit 170, the diagnosis unit 130 may exclude a lesion, which has been recognized by a user, from an area to be diagnosed. The diagnosis unit 130 may reduce the total computation required by not diagnosing an area where the lesion has been recognized by the user. The diagnosis unit 130 may reduce the amount of total computation based on one or more of a location of the lesion, information about movement of a probe, and a measuring range of the probe.

The guide information providing unit 150 may generate guide information to guide the probe from the present location toward a location of the detected lesion. If the lesion has been detected, but not recognized by the user, the user may move a probe away from the location of a detected lesion during a processing time of the diagnosis unit 130. The user may also move a probe away from the location of a detected lesion even where the lesion has been previously detected and recognized by the user. To enable a user to move the probe toward a location of a detected lesion, the guide information providing unit 150 generates guide information based on lesion information stored in the storage unit 170 and provides the guide information to the user.

The guide information may include at least one of a visual signal, an audio signal, or a tactile signal. For example, the guide information providing unit 150 may provide a visualized image for at least one of a moving path, a moving distance, a measured angle, or a measurement direction of the probe using the lesion location information or the information about movement of the probe, which are stored in the storage unit 170. In another non-exhaustive example, if a user moves the probe toward the detected lesion, the guide information providing unit 150 may guide the probe toward the detected lesion by generating a specific sound, such as, for example, an alarm, a voice, or an increasing volume of a sound or frequency as the probe moves closer to the detected lesion. In another non-exhaustive example, if the user moves the probe in a direction toward the detected lesion, the guide information providing unit 150 may guide the probe toward the detected lesion by generating a vibration or increasing the intensity of the vibration as the probe moves closer to the detected lesion. The guide information described above are only non-exhaustive illustrations, and other types of guide information are considered to be well within the scope of the present disclosure.

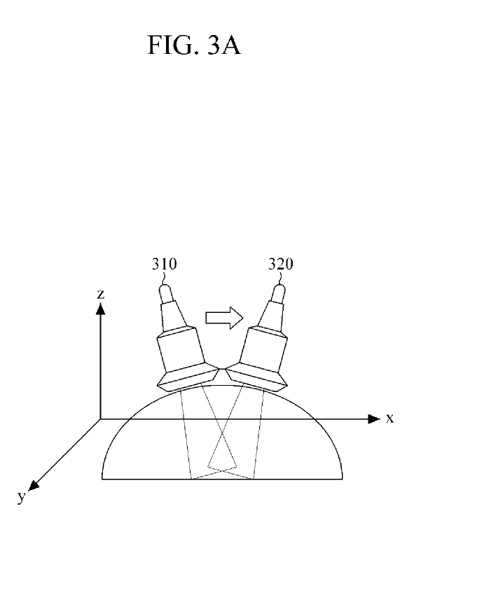

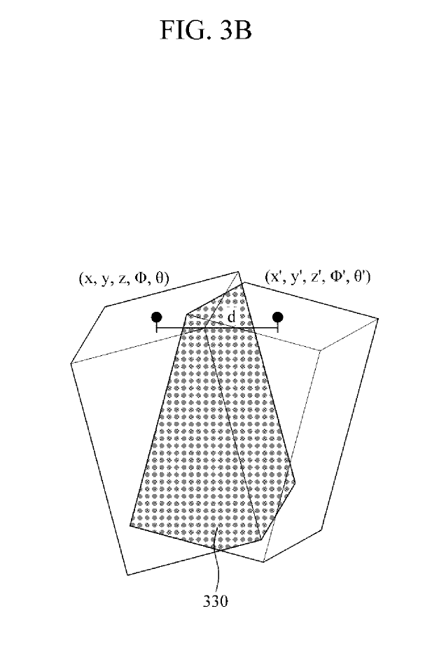

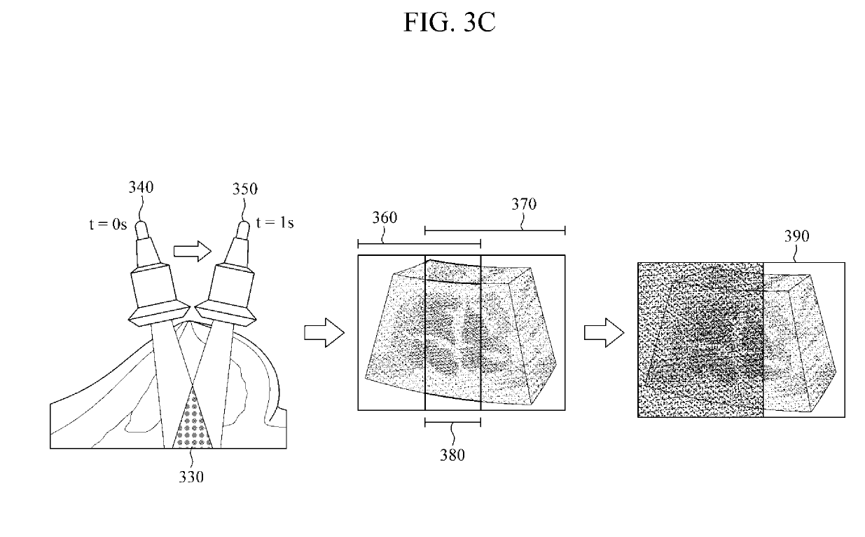

FIGS. 3A to 3C are diagrams illustrating examples for explaining location information of a lesion, information about movement of a probe and an overlapping area of an image. Referring to FIGS. 3A to 3C, when a probe is disposed at a location 310, a measurement direction φ, a measured angle θ, and coordinates (x, y, z) may be obtained using an accelerometer, such as, for example a gyroscope. If a user moves the probe toward a location 320, a measurement direction φ′, a measured angle θ′ and coordinates (x′, y′, z′) may be obtained. Here, the measurement angle may be a predetermined reference angle, and the coordinates may be absolute values measured with reference to an origin of absolute coordinates. The measurement angle may also be a relative angle measured in relation to a previous measurement angle, and the coordinates may be relative values measured in relation to previous coordinates. A relative distance (d) or a relative movement direction between the two measured locations may also be obtained using the measurement angle and the coordinates. If the probe includes an accelerometer, a moving path of the probe may also be tracked.

Referring to FIG. 3B, based on a measured direction, a measured angle, coordinates, and a measuring range of the probe at each of the locations 310 and 320, an overlapping area 330, which is measured more than once, may be calculated.

Referring to FIG. 3C, when diagnosing ailments, such as, for example, breast cancer or a thyroid gland, the diagnosis is performed by changing a measured angle and a measurement direction of a probe while moving the probe in a narrow range, and thus an overlapping area 330 is measured more than once. With reference to FIG. 3C, at time (t=0 s), an image 360 is created at measurement location 340, and at time (t=1 s), an image 370 is created at measurement location 350. As shown in FIG. 3C, the measurement range, measured angle, and measurement direction are different for images 360 and 370 and an overlapping area 380 is created between images 360 and 370. In this example, the total computation increases because the diagnosis unit 130 needs to diagnose the lesion more than once in the overlapping area 380. To decrease the amount of computation required and to increase responsiveness, the diagnosis unit 130 may diagnose only a new area 390 in image 370, and not repeat the diagnosis for the overlapping area 380, which is contained in both the image 370 and the previously input image 360.

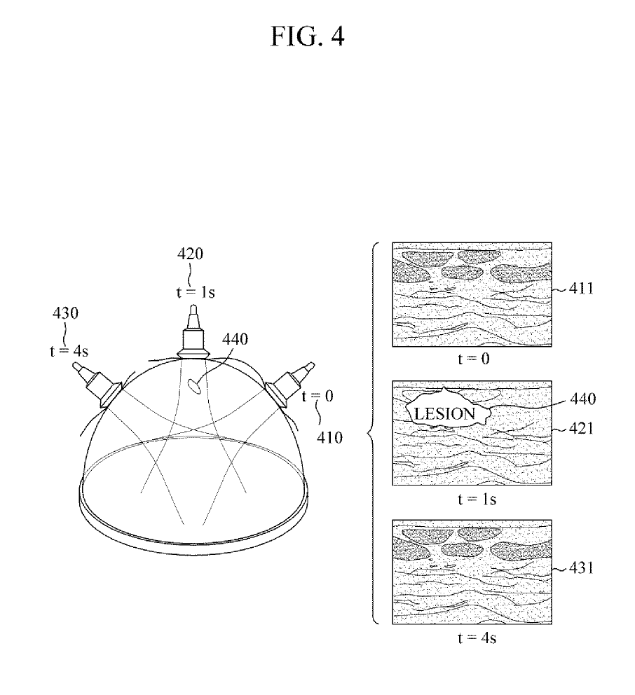

FIG. 4 is a diagram illustrating an example of generating a detection signal and guide information. Referring to FIG. 4, when diagnosing lesions in ultrasound medical images, a user recognizes a lesion by checking medical images generated in real time by a probe. The user may not recognize lesions if the user moves a probe too quickly, or if the user fails to continually concentrate on the ultrasound medical images, or if the user is not skilled at analyzing ultrasound medical images. In FIG. 4, a probe moves from measurement location 410 (at time t=0 s) to measurement location 420 (at time t=1 s) and to measurement location 430 (at time t=4 s). A lesion 440 is not detected from an ultrasound image 411 measured at a measurement location 410, but the lesion 440 is detected from an ultrasound image 421 measured at a location 420. If the user continues to move the probe without realizing that the lesion is detected, the user will fail to recognize the lesion because the lesion will not be detected from an ultrasound image 431 measured at a location 430. If the diagnosis unit 130 detects a lesion, a guide information providing unit 150 may instantly generate a detection signal to notify the user that a lesion is detected.

In a non-exhaustive example, the guide information providing unit 150 may continue to generate a detection signal until it receives a confirmation from the user that the detected lesion has been recognized. If the user continues to move a probe without recognizing the detection signal, the guide information providing unit 150 may continue to generate the detection signal until it receives a confirmation from a user through an input means (not illustrated) that the detected lesion is recognized.

In another non-exhaustive example, if a determination is made, based on lesion information stored in a storage unit 170, that a detected lesion is different from a previously detected lesion, the guide information providing unit 150 may generate a detection signal. On the other hand, if a determination is made, based on one or more of location information of the lesion stored in the storage unit 170, information about movement of a probe, or features of the lesion, that a detected lesion is identical to a previous detected lesion, the guide information providing unit 150 may not generate a detection signal.

In another non-exhaustive example, if a lesion is detected, a user may input a confirmation through an input means (not illustrated) that the detected lesion is recognized, and the storage unit 170 may store that the detected legion has been recognized by the user. Thereafter, if a determination is made, based on one or more of location information of a lesion stored in the storage unit 170, information about movement of a probe, features of the lesion, or whether the lesion has been recognized by the user, that a lesion, which has not been recognized by a user is re-detected, the guide information providing unit 150 may generate a detection signal. In another non-exhaustive example, if it is determined that a lesion is re-detected, which was previously recognized by the user but now has a different diagnosis, the guide information providing unit 150 may generate a detection signal.

A location of a lesion may change due to pressure, and the probe may include a pressure sensor to identify the lesion with a predetermined position margin of a location of a detected lesion.

Even if a user recognizes a detection signal, the probe may leave a location of the detected lesion since there is a difference between a point in time when a medical image is captured and a point in time when the diagnosis unit 130 diagnoses the medical image, and generates the detection signal. The guide information providing unit 150 may generate guide information to guide the probe from the present location, say 430, toward a location 420 of the detected lesion.

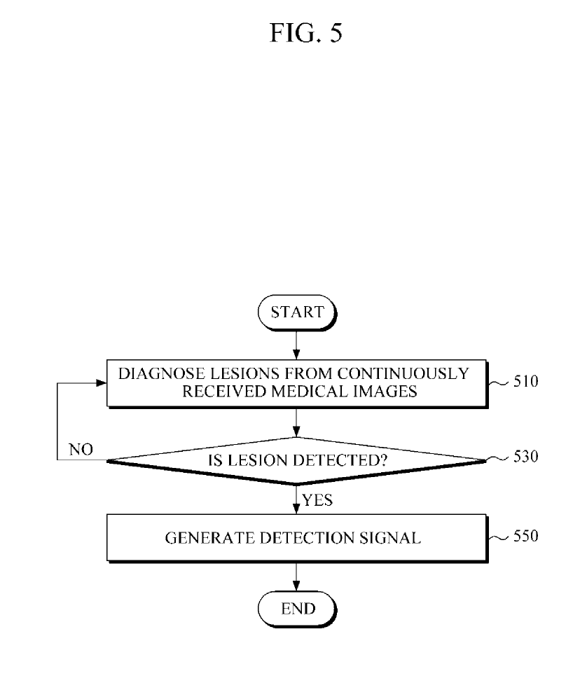

FIG. 5 is a diagram illustrating an example of a method for generating a detection signal. The operations in FIG. 5 may be performed in the sequence and manner as shown, although the order of some operations may be changed or some of the operations omitted without departing from the spirit and scope of the illustrative examples described. Many of the operations shown in FIG. 5 may be performed in parallel or concurrently. The description of FIGS. 1-4 is also applicable to FIG. 5, and thus will not be repeated here.

Referring to FIG. 5, in 510, a diagnosis unit 130 diagnoses lesions in medical images continuously received from a probe. The diagnosis unit 130 may perform a lesion diagnosis on each frame of a received medical image or on every predetermined number of frames by performing sampling of all the frames of the received medical image. One or more diagnosis unit 130 may be provided, and the medical images generated by a medical image generation unit 110 may be distributed to the multiple diagnosis units 130 to be diagnosed in parallel. In another non-exhaustive example, the diagnosis unit 130 may diagnose a non-overlapping area of the received medical images, thereby reducing the total amount of computation and increasing responsiveness.

In 530, if the diagnosis unit 130 detects a lesion, the guide information providing unit 150 generates a detection signal in 550. In another non-exhaustive example, if a user confirms that the detected lesion is recognized, the storage unit 170 may store whether the user has recognized the detected lesion.

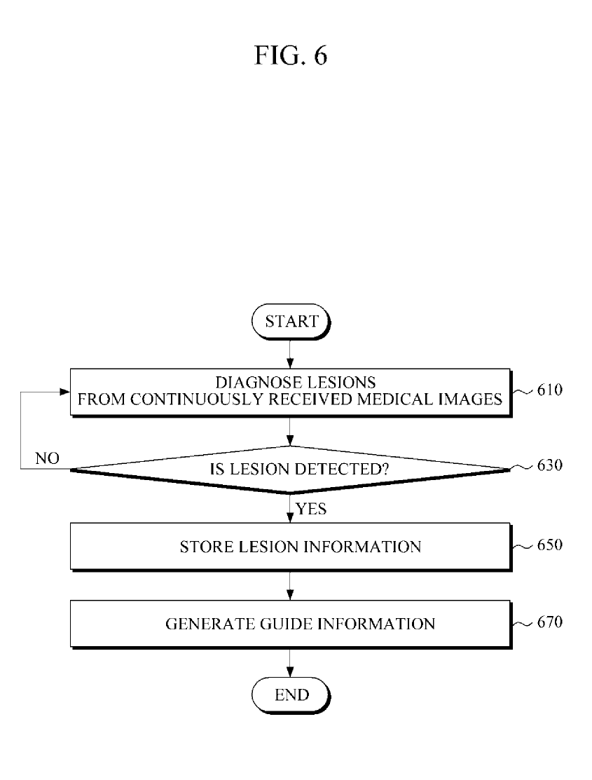

FIG. 6 is a diagram illustrating an example of a method for generating guide information. The operations in FIG. 6 may be performed in the sequence and manner as shown, although the order of some operations may be changed or some of the operations omitted without departing from the spirit and scope of the illustrative examples described. Many of the operations shown in FIG. 6 may be performed in parallel or concurrently. The description of FIGS. 1-5 is also applicable to FIG. 6, and thus will not be repeated here.

Referring to FIG. 6, in 610, a diagnosis unit 130 diagnoses lesions in medical images continuously received from a probe. In 630, if the diagnosis unit 130 detects a lesion, a storage unit 170 stores lesion information of the detected lesion in 650. The lesion information may include at least one of features of the lesion, diagnosis of the lesion, location information of the lesion, or information about movement of a probe after the lesion is detected. In 670, based on the stored lesion information, the guide information providing unit 150 may generate guide information to guide the probe from a present location toward a location of the detected lesion in 670.

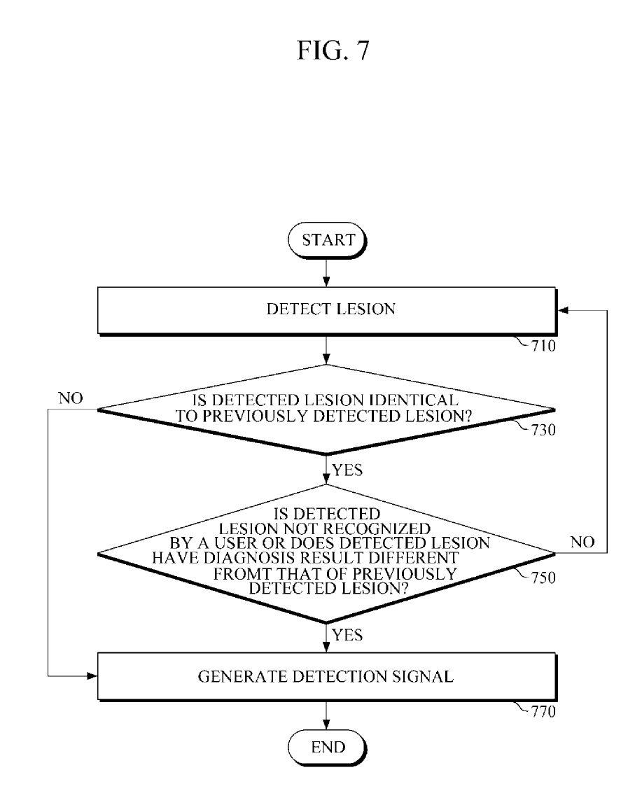

FIG. 7 is a diagram illustrating an example of a method for generating a detection signal when a lesion is detected again. The operations in FIG. 7 may be performed in the sequence and manner as shown, although the order of some operations may be changed or some of the operations omitted without departing from the spirit and scope of the illustrative examples described. Many of the operations shown in FIG. 7 may be performed in parallel or concurrently. The description of FIGS. 1-6 is also applicable to FIG. 7, and thus will not be repeated here.

Referring to FIG. 7, in 710, if a diagnosis unit 130 detects a lesion, in 730, a guide information providing unit 150 determines whether the detected lesion is identical to a previously detected lesion. In 730, based on the lesion information stored in the storage unit 170, the guide information providing unit 150 determines whether the detected lesion is identical to the previously detected lesion. In 770, if it is determined that the detected lesion is different from the previously detected lesion, the guide information providing unit 150 generates a detection signal.

In 750, it is determined whether the detected lesion is identical to the previously detected lesion but has not been recognized by the user, or if the detected lesion is identical to the previously detected lesion but its diagnosis is different from that of the previously detected lesion. If the user has not previously recognized the detected lesion or its diagnosis is different, then in 770, the guide information providing unit 150 generates a detection signal.

The lesion diagnosis apparatus, described above, notifies a user of a lesion being detected by analyzing a medical image, and provides a user with a location of the detected lesion, thereby reducing the possibility of overlooking any lesion and improving the user's convenience. In addition, the lesion diagnosis apparatus diagnosis lesions more rapidly by removing redundancies and minimizing the possibility of overlooking the diagnosis of a lesion.

The methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, WiFi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A lesion diagnosis apparatus comprising:
a diagnoser configured to diagnose lesions in images received from a probe; and
a guide information provider configured to notify a user that a lesion is detected and to provide guide information, when the lesion is detected.

2. The lesion diagnosis apparatus of claim 1, wherein the diagnoser is further configured to diagnose a non-overlapping area in an image using at least one of coordinates, a measuring range, a measured angle, or a measurement direction of the probe.

3. The lesion diagnosis apparatus of claim 1, further comprising:
a store configured to store lesion information when the lesion is detected, and the lesion information comprises at least one of features of the lesion, diagnosis of the lesion, location information of the lesion, or information about the movement of the probe after the lesion is detected.

4. The lesion diagnosis apparatus of claim 1, wherein the guide information guides the probe toward a location of the lesion, and the guide information comprises at least one of a moving path, a moving distance, a moving direction, or a measured angle of the probe.

5. The lesion diagnosis apparatus of claim 4, wherein the guide information is provided as at least one of an audio signal, a visual signal, or a tactile signal.

6. The lesion diagnosis apparatus of claim 1, wherein the guide information provider notifies the user that a lesion is detected by generating a detection signal and the detection signal comprises at least one of a visual signal, an audio signal, or a tactile signal.

7. The lesion diagnosis apparatus of claim 3, wherein, in response to a determination being made that the detected lesion is different from a previously detected lesion, the guide information provider is configured to generate a detection signal.

8. The lesion diagnosis apparatus of claim 3, further comprising:
an input means configured to receive a confirmation from the user that the detected lesion is recognized; and
the lesion information further comprises indication whether the user has recognized the detected lesion.

9. The lesion diagnosis apparatus of claim 8, wherein the guide information provider is configured to generate a detection signal when a lesion that has not been recognized by the user is re-detected.

10. The lesion diagnosis apparatus of claim 8, wherein the guide information provider is configured to generate a detection signal when a lesion is re-detected and its diagnosis is different from a previous diagnosis.

11. The lesion diagnosis apparatus of claim 3, wherein the features of the lesion comprises at least one of shape of the lesion, boundary of the lesion, posterior acoustic shadow of the lesion, echo pattern, microlobulations, hyperechoic halo, hypoechoic nodule, duct extension, compressibility, branching pattern, or density of the lesions.

12. The lesion diagnosis apparatus of claim 8, wherein the diagnoser is further configured to exclude the detected lesion that has been recognized by the user, from an area to be diagnosed.

13. A lesion diagnosis method comprising:
diagnosing lesions in images received from a probe;
generating, at a guide information provider, a detection signal to notify a user that the lesion is detected; and
providing guide information to guide the probe toward a location of the detected lesion.

14. The lesion diagnosis method of claim 13, wherein the diagnosing of lesions comprises diagnosing a non-overlapping area in a received image by using at least one of coordinates, a measuring range, a measured angle, or a measurement direction of the probe.

15. The lesion diagnosis method of claim 13, further comprising storing lesion information when the lesion is detected, and the lesion information comprises at least one of features of the lesion, diagnosis of the lesion, location information of the lesion, or information about the movement of the probe after the lesion is detected.

16. The lesion diagnosis method of claim 13, wherein the guide information comprises at least one of a moving path, a moving distance, a moving direction, or a measured angle of the probe.

17. The lesion diagnosis method of claim 16, wherein the providing of the guide information comprises providing the guide information as at least one of an audio signal, a visual signal, or a tactile signal.

18. The lesion diagnosis method of claim 13, wherein the detection signal comprising at least one of a visual signal, an audio signal, or a tactile signal.

19. The lesion diagnosis method of claim 15, wherein the generating of the detection signal comprises, generating a detection signal when the detected lesion is different from a previously detected lesion.

20. The lesion diagnosis method of claim 15, further comprising receiving a confirmation from the user that the detected lesion is recognized, and the lesion information further comprises indication whether the user has recognized the detected lesion.

21. The lesion diagnosis method of claim 20, wherein the generating of the detection signal comprises, generating a detection signal when a lesion that has been detected but not recognized by the user is re-detected.

22. The lesion diagnosis method of claim 20, wherein the generating of the detection signal comprises generating a detection signal when a lesion is re-detected and its diagnosis is different from a previous diagnosis.

23. The method of claim 15, wherein the features of the lesion comprises at least one of shape of the lesion, boundary of the lesion, posterior acoustic shadow of the lesion, echo pattern, microlobulations, hyperechoic halo, hypoechoic nodule, duct extension, compressibility, branching pattern, or density of the lesions.

24. The lesion diagnosis method of claim 20, wherein the diagnosing of the lesions comprises excluding the detected lesion that has been recognized by the user, from an area to be diagnosed.

25. A lesion diagnosis method comprising:
diagnosing lesions, in real-time, for images received from a probe; and
generating, at a guide information provider, a detection signal to notify a user that the lesion is detected.

26. The lesion diagnosis method of claim 25, wherein the diagnosing lesions in real-time comprises:
diagnosing lesions in a first image received from the probe;
identifying a non-overlapping area between the first image and a second image received from the probe; and
diagnosing lesions only in the non-overlapping area.

27. The lesion diagnosis method of claim 26, wherein the non-overlapping area is identified using at least one of coordinates, a measuring range, a measured angle, or a measurement direction of the probe.

28. The lesion diagnosis method of claim 26, further comprising:
providing guide information to guide the probe toward a location of the detected lesion; and
receiving a confirmation from the user that the detected lesion is recognized.

29. The lesion diagnosis method of claim 28, wherein the providing of the guide information comprises continuing to provide guide information until the user confirms that the detected lesion is recognized.

30. The lesion diagnosis method of claim 28, further comprising:
storing lesion information when the lesion is detected; and
the lesion information comprises at least one of features of the lesion, diagnosis of the lesion, location information of the lesion, information about the movement of the probe after the lesion is detected, or indication whether the user has recognized the detected lesion.

31. A lesion diagnosis apparatus comprising:
a diagnoser configured to receive an image captured by a probe at a first point in time, and to detect lesions in the image at a second point in time; and
a guide information provider configured to provide guide information to compensate for the movement of the probe between the first point in time and the second point in time.

32. The lesion diagnosis apparatus of claim 31, wherein the guide information guides the probe toward a location of the lesion, and the guide information comprises at least one of a moving path, a moving distance, a moving direction, or a measured angle of the probe.

33. The lesion diagnosis apparatus of claim 32, wherein the guide information is provided as at least one of an audio signal, a visual signal, or a tactile signal.

34. The lesion diagnosis apparatus of claim 33, wherein the guide information provider is further configured to generate an increasing volume of a sound or frequency as a user moves the probe closer to the detected lesion.

35. The lesion diagnosis apparatus of claim 33, wherein the guide information provider is further configured to increase the intensity of vibration of the probe as a user moves the probe closer to the detected lesion.
